(19) United States
(12) Patent Application Publication
(10) Pub. No.: US 2022/0347137 A1
(43) Pub. Date: Nov. 3, 2022

(54) COMPOSITIONS AND METHODS OF TREATING ACNE AND PHOTOAGING

(71) Applicant: Curology, Inc., San Francisco, CA (US)

(72) Inventor: David LORTSCHER, San Francisco, CA (US)

(21) Appl. No.: 17/722,236

(22) Filed: Apr. 15, 2022

Related U.S. Application Data
(63) Continuation of application No. PCT/US2020/055725, filed on Oct. 15, 2020.
(60) Provisional application No. 63/065,156, filed on Aug. 13, 2020.
(60) Provisional application No. 62/915,832, filed on Oct. 16, 2019.

(51) Int. Cl. A61K 31/194; A61K 31/07; A61Q 19/08; A61P 17/10; A61K 31/455

(52) U.S. Cl. CPC A61K 31/194; A61K 31/07; A61Q 19/08; A61P 17/10; A61K 31/455

(57) ABSTRACT

Pharmaceutical compositions for treating acne, photoaging, and uneven pigmentation comprising three distinct pharmaceutical ingredients are described. Methods for the treatment of acne, photoaging, and uneven pigmentation using the compositions are also described.

### TECHNICAL FIELD

The present invention relates generally to the field of dermatology and more specifically to compositions and methods for the treatment of acne, uneven pigmentation, and photoaging.

### BACKGROUND

Acne occurs in greater than 90% of the population at some point in their lives. Although it is primarily considered a disorder of the teenage years, many people (and especially females) suffer from acne during adulthood. Acne (also known as acne vulgaris) is a long-term skin condition that is caused by: 1) plugging of hair follicles by abnormally keratinized cells; 2) microbial colonization of the follicle; 3) inflammation; and 4) increased oil production associated with circulating hormones.

Photoaging occurs naturally as our skin is exposed to the sun's ultraviolet rays, and the first signs of photoaging (including fine wrinkles and hyperpigmentation) typically appear between the ages of 20 and 35. While sun protection is key to minimizing photoaging, there are also various topical treatments which have proven to be efficacious for treating and preventing photoaging.

The desire to treat acne can coexist with the desire to treat and/or prevent photoaging. While the application of multiple dermatologic products is an option currently employed by many patients, the existence of a single, efficacious, stable composition would offer benefits in convenience and adherence.

However, there are numerous difficulties in formulating a single, efficacious, stable composition to treat or prevent acne and photoaging.

For example, the successful treatment of acne, alone, typically involves using two different agents with complementary mechanisms of action. The common categories are comedolytics (which help keratinization and thus prevent clogged pores) and antimicrobials (which generally target the acne-causing bacterium Propionibacterium acnes or P. acnes). Thus, the successful treatment of acne and photoaging together would typically require three or more active ingredients, which may require different vehicles, different frequencies of application, and different methods of application.

Another difficulty inherent in creating a combined formulation is that many anti-acne ingredients inactivate other anti-acne ingredients. For example, benzoyl peroxide inactivates tretinoin, erythromycin, and hydroquinone; tretinoin inactivates erythromycin; and benzoyl peroxide can lead to oxidation of zinc pyrithione. There are likely to be many more similar interactions that are not yet described in the dermatology literature.

When it is desired to use anti-photoaging ingredients or uneven pigmentation ingredients in addition to anti-acne ingredients, additional interactions can arise. When a patient is using benzoyl peroxide (for acne) they should avoid using it at the same time as hydroquinone (used for short-term treatment of photoaging), as the combination can lead to staining of the skin. As another example, niacinamide (a vitamin B3 derivative that can be used as an anti-acne ingredient and as an anti-photoaging ingredient) should not be used with ascorbic acid (the naturally occurring form of vitamin C), as the former can inactivate the latter ingredient. In addition, many photoaging or uneven pigmentation treatments cannot be used long-term because they contain steroids or a bleaching agent (hydroquinone) with potential undesirable side effects.

Thus, for patients receiving treatment for both acne and photoaging, their treatments typically are not included in the same formulation, and additionally, the patients are often instructed to use their individual formulations at different times of day, significantly decreasing the convenience of treatment.

An additional difficulty in formulating a once-daily composition for the treatment of acne and photoaging is that the majority of ingredients for each of these purposes are typically applied to the skin twice daily. These ingredients that are typically applied twice daily include clindamycin, azelaic acid, dapsone, adapalene, benzoyl peroxide, erythromycin, hydroquinone, niacinamide, ascorbic acid, magnesium ascorbyl phosphate, zinc pyrithione, and others. Even for treatment of acne alone, once-daily treatments are not yet the norm because of the potential inactivation of one anti-acne compound by another anti-acne compound and using two different agents with different mechanisms of action often requiring different formulations.

Also, a method to treat both acne and photoaging requires a collection of active ingredients that are stable and efficacious in the same vehicle. In formulating a vehicle of inactive ingredients to use along with active ingredient(s), one must account for texture, color, scent, method of application, pH, water solubility, alcohol solubility, stability of the active ingredients, and the presence or absence of interactions between the active ingredient(s) and the inactive ingredients. Thus, for both acne and photoaging to be treated with a single treatment is a significant advance over most current methodologies. A once-daily composition and method of treatment would be desirable because a once-daily composition increases patient adherence and lowers cost.

### BRIEF DESCRIPTION OF THE FIGURES

The foregoing and other objects of the present disclosure, the various features thereof, as well as the disclosure itself may be more fully understood from the following description, when read together with the accompanying drawings in which:

FIG. 1 shows exemplary pharmaceutical compositions for the treatment of acne and photoaging, wherein the abbreviations used are as follows: azelaic acid (AzA); niacinamide (Nia); tretinoin (Tret); metronidazole (Met); tranexamic acid (TxA); and zinc pyrithione (Zinc).

### SUMMARY

The disclosure provides pharmaceutical compositions, methods, and kits for the treatment of skin disorders.

The disclosure provides, in one aspect, a composition comprising metronidazole, azelaic acid, and zinc pyrithione, or pharmaceutically acceptable salts thereof.

In another aspect, the disclosure provides a composition comprising between 0.5% w/w and 2% w/w metronidazole, between 4% w/w and 6% w/w azelaic acid, and between 0.1% w/w and 0.5% w/w zinc pyrithione, or pharmaceutically acceptable salts thereof.

The disclosure provides, in one aspect, a composition comprising about 1% w/w metronidazole, about 5% w/w azelaic acid, and about 0.25% w/w zinc pyrithione, or pharmaceutically acceptable salts thereof.

In yet another aspect, the disclosure provides a composition azelaic acid, tranexamic acid, and niacinamide, or pharmaceutically acceptable salts thereof.

The disclosure provides, in one aspect, a composition comprising between 4% w/w and 6% w/w azelaic acid, between 1% w/w and 4% w/w tranexamic acid, and between 2% w/w and 6% w/w niacinamide, or pharmaceutically acceptable salts thereof.

In yet another aspect, the disclosure provides a composition comprising about 5% w/w azelaic acid, about 2% w/w tranexamic acid, and about 4% w/w niacinamide, or pharmaceutically acceptable salts thereof.

In another aspect, the disclosure provides a composition comprising tretinoin, tranexamic acid, and niacinamide, or pharmaceutically acceptable salts thereof.

In yet another aspect, the disclosure provides a composition comprising between 0.001% w/w and 0.2% w/w tretinoin, between 1% w/w and 6% w/w tranexamic acid, and between 2% w/w and 6% w/w niacinamide, or pharmaceutically acceptable salts thereof.

In another aspect, the disclosure provides a composition comprising about 0.003% w/w tretinoin, from about 2% to about 5% w/w tranexamic acid, and about 4% w/w niacinamide, or pharmaceutically acceptable salts thereof. In some embodiments the composition comprises about 2% w/w tranexamic acid. In some embodiments the composition comprises about 5% w/w tranexamic acid.

In yet another aspect, the disclosure provides a composition comprising about 0.005% w/w tretinoin, between 2% w/w and 5% w/w tranexamic acid, and about 4% w/w niacinamide, or pharmaceutically acceptable salts thereof. In some embodiments the composition comprises about 2% w/w tranexamic acid. In some embodiments the composition comprises about 5% w/w tranexamic acid.

In still another aspect, the disclosure provides a composition comprising about 0.007% w/w tretinoin, between 2% w/w and 5% w/w tranexamic acid, and about 4% w/w niacinamide, or pharmaceutically acceptable salts thereof. In some embodiments the composition comprises about 2% w/w tranexamic acid. In some embodiments the composition comprises about 5% w/w tranexamic acid.

In still another aspect, the disclosure provides a composition comprising about 0.009% w/w tretinoin, between 2% w/w and 5% w/w tranexamic acid, and about 4% w/w niacinamide, or pharmaceutically acceptable salts thereof. In some embodiments the composition comprises about 2% w/w tranexamic acid. In some embodiments the composition comprises about 5% w/w tranexamic acid.

In another aspect, the disclosure provides a composition comprising about 0.012% w/w tretinoin, between 2% w/w and 5% w/w tranexamic acid, and about 4% w/w niacinamide, or pharmaceutically acceptable salts thereof. In some embodiments the composition comprises about 2% w/w tranexamic acid. In some embodiments the composition comprises about 5% w/w tranexamic acid.

In still another aspect, the disclosure provides a composition comprising about 0.015% w/w tretinoin, between 2% w/w and 5% w/w tranexamic acid, and about 4% w/w niacinamide, or pharmaceutically acceptable salts thereof. In some embodiments the composition comprises about 2% w/w tranexamic acid. In some embodiments the composition comprises about 5% w/w tranexamic acid.

In another aspect, the disclosure provides a composition comprising about 0.02% w/w tretinoin, between 2% w/w and 5% w/w tranexamic acid, and about 4% w/w niacinamide, or pharmaceutically acceptable salts thereof. In some embodiments the composition comprises about 2% w/w tranexamic acid. In some embodiments the composition comprises about 5% w/w tranexamic acid.

In yet another aspect, the disclosure provides a composition comprising about 0.035% w/w tretinoin, between 2% w/w and 4% w/w tranexamic acid, and about 4% w/w niacinamide, or pharmaceutically acceptable salts thereof. In some embodiments the composition comprises about 2% w/w tranexamic acid. In some embodiments the composition comprises about 5% w/w tranexamic acid.

In still another aspect, the disclosure provides a composition comprising about 0.05% w/w tretinoin, between 2% w/w and 5% w/w tranexamic acid, and about 4% w/w niacinamide, or pharmaceutically acceptable salts thereof. In some embodiments the composition comprises about 2% w/w tranexamic acid. In some embodiments the composition comprises about 5% w/w tranexamic acid.

In another aspect, the disclosure provides a composition comprising about 0.07% w/w tretinoin, between 2% w/w and 5% w/w tranexamic acid, and about 4% w/w niacinamide, or pharmaceutically acceptable salts thereof. In some embodiments the composition comprises about 2% w/w tranexamic acid. In some embodiments the composition comprises about 5% w/w tranexamic acid.

In yet another aspect, the disclosure provides a composition comprising about 0.1% w/w tretinoin, between 2% w/w and 5% w/w tranexamic acid, and about 4% w/w niacinamide, or pharmaceutically acceptable salts thereof. In some embodiments the composition comprises about 2% w/w tranexamic acid. In some embodiments the composition comprises about 5% w/w tranexamic acid.

In yet another aspect, the disclosure provides a composition comprising about 0.14% w/w tretinoin, between 2% w/w and 5% w/w tranexamic acid, and about 4% w/w niacinamide, or pharmaceutically acceptable salts thereof. In some embodiments the composition comprises about 2% w/w tranexamic acid. In some embodiments the composition comprises about 5% w/w tranexamic acid.

In another aspect, the disclosure provides a composition comprising tretinoin, tranexamic acid, and azelaic acid, or pharmaceutically acceptable salts thereof.

In yet another aspect, the disclosure provides a composition comprising about 0.025% w/w tretinoin, about 8% w/w azelaic acid, and between 2% w/w and 5% w/w tranexamic acid, or pharmaceutically acceptable salts thereof. In some embodiments this composition comprises about 2% tranexamic acid. In some embodiments this composition comprises about 5% w/w tranexamic acid.

In still another aspect, the disclosure provides composition comprising about 0.003% w/w tretinoin, about 2% w/w azelaic acid, and about 4% w/w niacinamide, or pharmaceutically acceptable salts thereof.

In another aspect, the disclosure provides a composition comprising about 0.005% w/w tretinoin, about 2% w/w azelaic acid, and about 4% w/w niacinamide, or pharmaceutically acceptable salts thereof.

In an additional aspect, the disclosure provides a composition comprising about 0.007% w/w tretinoin, about 2% w/w azelaic acid, and about 4% w/w niacinamide, or pharmaceutically acceptable salts thereof.

In another aspect, the disclosure provides a composition comprising about 0.010% w/w tretinoin, about 2% w/w azelaic acid, and about 4% w/w niacinamide, or pharmaceutically acceptable salts thereof.

In yet another aspect, the disclosure provides a composition comprising about 0.012% w/w tretinoin, about 5% w/w azelaic acid, and about 4% w/w niacinamide, or pharmaceutically acceptable salts thereof.

In an additional aspect, the disclosure provides a composition comprising about 0.015% w/w tretinoin, about 5% w/w azelaic acid, and about 4% w/w niacinamide, or pharmaceutically acceptable salts thereof.

In another aspect, the disclosure provides a composition comprising about 0.1% w/w tretinoin, about 5% w/w azelaic acid, and about 4% w/w niacinamide, or pharmaceutically acceptable salts thereof.

In some embodiments, the compositions according to the disclosure are for topical administration.

In some embodiments, the composition further comprises a pharmaceutically acceptable vehicle. In certain embodiments, the composition further comprises at least one pharmaceutically acceptable vehicle, at least one emulsifier, at least one glycol, and at least one preservative.

In some embodiments, the composition is used for the treatment of acne, photoaging, and/or uneven pigmentation.

In another aspect, the disclosure provides methods for the treatment of acne, photoaging, and/or uneven pigmentation in a subject in need thereof, comprising administering to the skin of the subject a composition according to the disclosure. In some embodiments, the composition has a pH of between 3.5 and 6.0.

In another aspect, the disclosure provides a kit comprising one of the compositions of the disclosure, a sealed container for housing the composition, and instructions for use. In some embodiments, the composition of the kit is administered topically. In some embodiments, the composition of the kit has a pH of between 3.5 and 6.0.

In yet another aspect, the disclosure provides a method for treating acne or photoaging in a subject in need thereof comprising:
a) administering a therapeutically effective amount of a first composition to the skin of the subject, wherein the first composition comprises niacinamide, tranexamic acid, and tretinoin, or pharmaceutically acceptable salts thereof; and
b) administering a therapeutically effective amount of a second composition to the skin of the subject after a first interval after administering the first composition, wherein the second composition comprises niacinamide, tranexamic acid, and tretinoin, or pharmaceutically acceptable salts thereof, and wherein the composition comprises a higher concentration of tretinoin or tranexamic acid or both than the first composition. In some embodiments, the method further comprises administering a therapeutically effective amount of a third composition to the skin of the subject after a second interval after administering the second composition, wherein the third composition comprises niacinamide, tranexamic acid, and tretinoin, or pharmaceutically acceptable salts thereof, and wherein the composition comprises a higher concentration of tretinoin or tranexamic acid or both than the second composition. In some embodiments, the first composition comprises about 2% w/w tranexamic acid and the second and/or third composition comprises about 5% w/w tranexamic acid. In some embodiments, the first and second compositions comprise between 0.003% w/w and 0.14% w/w tretinoin. In some embodiments, the first, second, and third compositions comprise between 0.003% w/w and 0.14% w/w tretinoin. In some embodiments, the first composition comprises a percentage of tretinoin selected from the group consisting of about 0.003% w/w, about 0.005% w/w, about 0.007% w/w, about 0.009% w/w, about 0.012% w/w, about 0.015% w/w, about 0.02% w/w, about 0.035% w/w, about 0.05% w/w, about 0.07% w/w, and about 0.100% w/w. In some embodiments, the second composition comprises a percentage of tretinoin selected from the group consisting of about 0.005% w/w, about 0.007% w/w, about 0.009% w/w, about 0.012% w/w, about 0.015% w/w, about 0.02% w/w, about 0.035% w/w, about 0.05% w/w, about 0.07% w/w, about 0.1% w/w, and about 0.14% w/w. In some embodiments, the third composition comprises a percentage of tretinoin selected from the group consisting of about 0.005% w/w, about 0.007% w/w, about 0.009% w/w, about 0.012% w/w, about 0.015% w/w, about 0.02% w/w, about 0.035% w/w, about 0.05% w/w, about 0.07% w/w, about 0.1% w/w, and about 0.14% w/w. In some embodiments, the skin of the subject is evaluated during the first interval. In some embodiments, if adverse skin reactions are not observed while evaluating the skin of the subject during the first interval, then a higher percentage of tretinoin or tranexamic acid is used in the second composition than was used in the first composition. In some embodiments, the skin of the subject is evaluated during the second interval. In some embodiments, if adverse skin reactions are not observed while evaluating the skin of the subject during the second interval, then a higher percentage of tretinoin or tranexamic acid is used in the third composition than was used in the second composition. In some embodiments, evaluating the skin of the subject comprises evaluating skin by profilometry; evaluating skin tone; evaluating skin color; evaluating skin firmness; evaluating skin elasticity; evaluating skin hydration; and evaluating skin aging by visual assessments. In some embodiments, the first or second interval is between about 2 and about 6 weeks. In some embodiments, the first or second interval is about 4 weeks.

In yet another aspect, the disclosure provides a method for treating acne or photoaging in a subject in need thereof comprising:
a) administering a therapeutically effective amount of a first composition to the skin of the subject, wherein the first composition comprises niacinamide, azelaic acid, and tretinoin or pharmaceutically acceptable salts thereof; and
b) administering a therapeutically effective amount of a second composition to the skin of the subject after a first interval after administering the first composition, wherein the second composition comprises niacinamide, azelaic acid, and tretinoin, or pharmaceutically acceptable salts thereof, and wherein the composition comprises a higher concentration of tretinoin or azelaic acid or both than the first composition. In some embodiments, the method further comprises administering a therapeutically effective amount of a third composition to the skin of the subject after a second interval after administering the second composition, wherein the third composition comprises niacinamide, azelaic acid, and tretinoin, or pharmaceutically acceptable salts thereof, and wherein the composition comprises a higher concentration of tretinoin or azelaic acid than the second composition. In some embodiments, the first composition comprises about 2% w/w azelaic acid and the second and/or third composition comprises about 5% w/w azelaic acid. In some embodiments, the first and second compositions comprise between 0.003% w/w and 0.140% w/w tretinoin. In some embodiments, the first, second, and third compositions comprises between 0.003% w/w and 0.140% w/w tretinoin. In some embodiments, the first composition comprises a percentage of tretinoin selected from the group consisting of about 0.003% w/w, about 0.005% w/w, about 0.007% w/w, about 0.010% w/w, about 0.012% w/w, about 0.015% w/w, about 0.02% w/w, about 0.035% w/w, about 0.05% w/w, about 0.070% w/w, and about 0.100% w/w. In some embodiments, the second composition comprises a percentage of tretinoin selected from the group consisting of about 0.005% w/w, about 0.007% w/w, about 0.01% w/w, about 0.012% w/w, about 0.015% w/w, about 0.02% w/w, about 0.035% w/w, about 0.05% w/w, about 0.07% w/w, about 0.1% w/w, and about 0.140% w/w. In some embodiments, the third composition comprises a percentage of tretinoin selected from the group consisting of about 0.005% w/w, about 0.007% w/w, about 0.01% w/w, about 0.012% w/w, about 0.015% w/w, about 0.02% w/w, about 0.035% w/w, about 0.05% w/w, about 0.070% w/w, about 0.1% w/w, and about 0.140% w/w. In some embodiments, the method further comprises evaluating the skin of the subject during the first interval. In some embodiments, if adverse skin reactions are not observed while evaluating the skin of the subject during the first interval, then a higher percentage of tretinoin or tranexamic acid is used in the second composition than was used in the first composition. In some embodiments, the method further comprises evaluating the skin of the subject during the second interval. In some embodiments, if adverse skin reactions are not observed while evaluating the skin of the subject during the second interval, then a higher percentage of tretinoin or tranexamic acid is used in the third composition than was used in the second composition. In some embodiments, evaluating the skin of the subject comprises evaluating skin by profilometry; evaluating skin tone; evaluating skin color; evaluating skin firmness; evaluating skin elasticity; evaluating skin hydration; and evaluating skin aging by visual assessments. In some embodiments, the first interval or second interval is between 2 and 6 weeks. In some embodiments, the first interval or second interval is about 4 weeks.

### DETAILED DESCRIPTION

The disclosures of these patents, patent applications, and publications in their entireties are hereby incorporated by reference into this application in order to more fully describe the state of the art as known to those skilled therein as of the date of the invention described and claimed herein. The instant disclosure will govern in the instance that there is any inconsistency between the patents, patent applications, and publications and this disclosure.

The disclosure provides pharmaceutical compositions, methods, and kits for the treatment of skin disorders.

### A. Terms, Definitions and Abbreviations

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

As used herein and unless otherwise expressly noted or required by the context, all percentages refer to percentages by weight (wt-%) of the total composition (w/w).

As used herein in connection with a measured quantity, for example weight, “about” refers to that variation in the measured quantity as would be expected by one skilled in the art exercising a level of care commensurate with the objective of the measurement and the equipment used, and includes uncertainties that may be introduced by mathematical rounding errors. In certain embodiments, “about” means plus or minus 5% of the recited amount.

As used herein an “anti-acne” compound is a compound that treats acne, for example, reducing the amount of acne. Anti-acne compounds include, but are not limited to, comedolytics (which help keratinization and thus prevent clogged pores), antibiotics (which can target the acne-causing bacterium Propionibacterium acnes (P. acnes) or the acne-causing mite Demodex folliculorum), and anti-inflammatory compounds (which have a direct effect on inflammation independent of any comedolytic or antibiotic effects). Non-limiting examples of comedolytics include alpha hydroxy acids (e.g., glycolic acid, lactic acid, and salicylic acid), retinoids (e.g., tretinoin and isotretinoin), and saturated dicarboxylic acids (e.g., suberic acid, azelaic acid, and sebacic acid). Non-limiting examples of antibiotics include cephalosporins (e.g., cefoxitin, ceftazidime, and cefepime), nitroimidazole (e.g., metronidazole), lincosamides (e.g., clindamycin and lincomycin), macrolides (e.g., erythromycin and azithromycin), pleuromutillins (e.g., retapamulin), metal complexes (e.g., zinc pyrithione, zinc methoxazole, and zinc sulfathiazole), penicillins (e.g., amoxicillin, ampicillin, and carbenicillin), fluoroquinolones (e.g., ciprofloxacin, clinafloxacin, ofloxacin, and trovafloxacin), retinoids (e.g., tretinoin), saturated dicarboxylic acids (e.g., suberic acid, azelaic acid, and sebacic acid), sulfonamides (e.g., sulfamethizole, sulfamethoxazole, sulfisoxazole), sulfones (e.g., dapsone or diaminodiphenyl sulfone), and tetracyclines (e.g., doxycycline and minocycline). Non-limiting examples of an anti-inflammatory compound include lincosamides (e.g., clindamycin and lincomycin), niacinamide (also known as nicotinamide and pyridine-3-corboxamide), retinoids (e.g., tretinoin), and saturated dicarboxylic acids (e.g., suberic acid, azelaic acid, and sebacic acid). For example, anti-acne compounds include metronidazole, azelaic acid, niacinamide, tretinoin, and zinc pyrithione, or a pharmaceutically acceptable salt thereof.

Saturated dicarboxylic acids can act as comedolytics and as antibiotics. Although azelaic acid is a useful anti-acne compound for use in those embodiments of the present disclosure in which an anti-acne compound is included, other saturated dicarboxylic acids may also be used, including suberic acid and sebacic acid. Azelaic acid (also known as nonanedioic acid) is an external treatment for, for example, acne, rosacea, melasma, and postinflammatory hyperpigmentation. Azelaic acid is also used as an antifungal.

Nitroimidazoles can act as antibiotics and anti-inflammatory agents. For example, metronidazole exhibits both antibiotic and anti-inflammatory activity. Metronidazole has antibiotic activity against Demodex folliculorum, a mite that lives on the skin that may play a role in both acne and rosacea.

Retinoids are well known to those skilled in the art of formulating topical dermatological compositions. Retinoids exhibit the pharmacological activity of all trans retinol and share, as a common structural feature, a β-ionone-type ring (2,6,6-trimethylcyclohen-1-ene) having a multiply unsaturated alkyl side chain at the 1 position of the ring. Tretinoin (also known all-trans retinoic acid) is the carboxylic acid form of vitamin A. Tretinoin, as well as other retinoid derivatives, such as but not limited to, retinol, adapalene, isotretinoin, alitretinoin, etretinate, acitretin, bexarotene, or tazarotene can also be used as anti-acne or anti-aging compounds.

Some metal complexes, including zinc pyrithione, have antibiotic effects. Although zinc pyrithione (also known as bis(2-pyridylthio)zinc 1,1′-dioxide) is a useful anti-acne compound for use in those embodiments of the present disclosure in which an anti-acne compound is included, other metal complexes can also be used, including zinc methoxazole and zinc sulfathiazole. Zinc pyrithione is also used as an antifungal. Zinc pyrithione is used to treat and prevent UV-induced skin damage and may also treat hyperpigmentation such as melasma.

The term “anti-inflammatory” compound for the purposes of the present disclosure refers to a compound that reduces certain signs of inflammation and may treat inflammatory acne (e.g., papules, pustules, nodules, and cysts) independent of any comedolytic or antimicrobial effects. For example, anti-inflammatory compounds include azelaic acid, niacinamide, and tretinoin.

As used herein an “anti-photoaging” compound is a compound that treats photoaging, for example, by reducing the amount of fine wrinkles or of hyperpigmentation. Anti-photoaging compounds include, but are not limited to, antioxidants (e.g., vitamins or vitamin derivatives including, but not limited to, niacinamide and ascorbyl phosphate, ascorbyl 6 palmitate, isostearyl 2-0 L-ascorbyl phosphate, and ascorbic acid sulfate), tyrosinase inhibitors (e.g., 4-n-butylresorcinol, azelaic acid, and kojic acid), and skin lightening agents (e.g., tranexamic acid). Tranexamic acid has skin lightening effects and can be used to treat hyperpigmentation, including hyperpigmentation that results from exposure to ultraviolet light and melasma. Exemplary anti-photoaging compounds include azelaic acid, niacinamide, and tranexamic acid, or a pharmaceutically acceptable salt thereof (e.g., magnesium ascorbyl phosphate and sodium ascorbyl phosphate).

The term “antioxidant” for the purposes of the present disclosure refers to a chemical substance that is added to a pharmaceutical composition to treat or to prevent photoaging, for example, by inhibiting the oxidation of molecules that are present in skin or dermis of a subject. Vitamins and vitamin derivatives are well known to those skilled in the art of formulating topical dermatological compositions. Certain vitamin derivatives have increased stability over the naturally occurring form of the vitamin For example, some vitamin E derivatives, including tocopheryl acetate, are more stable than the naturally occurring tocopherol (vitamin E), and some vitamin C derivatives, including ascorbyl phosphate, ascorbyl 6 palmitate, isostearyl 2-O L-ascorbyl phosphate, and ascorbic acid sulfate, or pharmaceutically acceptable salts thereof, are more stable than the naturally occurring L-ascorbic acid or ascorbate (vitamin C). Vitamin C is the most abundant antioxidant in the skin and is a cofactor in collagen production. Niacinamide is a form of vitamin B3 that fights acne via anti-inflammatory properties and has anti-aging effects.

The term “tyrosinase inhibitor” for the purposes of the present disclosure refers to a chemical compound that is added to a pharmaceutical composition to treat or to prevent photoaging, for example, by reducing the production of melanin by binding to tyrosinase present in skin or dermis of a subject. Tyrosinase is a copper-containing oxidase that catalyzes the first two steps in the production of melanin. Overproduction of melanin can lead to hyperpigmentation. Although azelaic acid is a useful anti-photoaging compound for use in those embodiments of the present disclosure in which an anti-photoaging compound is included, other tyrosinase inhibitor can also be used, including 4-n-butylresorcinol and kojic acid.

An “inactive ingredient” is compatible with the other ingredients of the formulation and not injurious to the patient or to the subject. Non-limiting examples of inactive ingredients include a preservative, a thickening agent, a vehicle, and a vitamin derivative.

The term “preservative” for the purposes of the present invention refers to a chemical substance that is added to a pharmaceutical composition to prevent the pharmaceutical composition from deterioration, decomposition or degradation or to substantially reduce or decelerate the degree and/or the speed of such deterioration, decomposition or degradation. Non-limiting examples of preservatives include benzoate, ethylhexylglycerin, methyl benzoate, methyl paraben, phenoxyethanol, propionic acid, propyl paraben, and pharmaceutically acceptable salts thereof.

An antioxidant can also be a preservative that contributes to the long-term storage and stability of the composition because of its function as a free radical scavenger. Non-limiting examples of antioxidant preservatives include butylated hydroxytoluene (BHT), butylated hydroxyanisole (BHA), tocopheryl acetate, ascorbic acid, ascorbyl palmitate, lecithin, norhydroguaiaretic acid, propyl gallate, a-tocopherol, sodium bisulfite, cysteine, sodium metabisulfite, thioglycerol, thioglycolic acid, thiomersal, and pharmaceutically acceptable salts thereof.

The term “vehicle” refers to a substance that serves as a carrier, whether diluent or excipient, for improving the efficiency of delivery and the effectiveness of a pharmaceutical composition. The phrase “pharmaceutically acceptable vehicle” is art recognized and includes a pharmaceutically acceptable material, composition or vehicle, suitable for administering compounds of the present invention to mammals. The vehicles include liquid or solid filler, diluent, excipient, solvent or encapsulating material, involved in carrying or transporting the subject agent from one organ, or portion of the body, to another organ, or portion of the body. Each vehicle must be “acceptable” in the sense of being compatible with the other ingredients of the formulation and not injurious to the patient or to the subject. Some examples of materials which can serve as pharmaceutically acceptable vehicles include: water; aloe vera leaf juice; aloe barbadensis leaf juice; emulsifiers or thickening agents, such as carbomer, cetearyl alcohol, cetyl alcohol, glyceryl stearate, stearic acid, xanthan gum, C13-14 isoparaffin, caprylic/capric triglyceride, polyacrylamide, and viscous liquids; sugars, such as lactose, glucose and sucrose; starches, such as corn starch and potato starch; maltodextrin; cellulose, and its derivatives, such as sodium carboxymethyl cellulose, ethyl cellulose and cellulose acetate; powdered tragacanth; malt; gelatin; talc; excipients, such as cocoa butter, myristyl myristate, Shea butter, and suppository waxes; oils, such as acai palm fruit oil, calendula flower oil, corn oil, cottonseed oil, jojoba seed oil, olive oil, passion fruit seed oil, peanut oil, rice bran oil, safflower oil, sesame oil, soybean oil, and sweet almond seed oil; glycols, such as propylene glycol and triethylene glycol; polyols, such as glycerin, vegetable glycerin, sorbitol, mannitol and polyethylene glycol (e.g., ceteareth-20, laureth-7, and PEG-100 myristate); esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations.

As used herein, the term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are useful. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

### B. Compositions

The disclosure provides a pharmaceutical composition for the treatment of skin disorders, such as a topically administered composition. In some aspects, the pharmaceutical composition comprises three distinct pharmaceutical ingredients, which may be supplied in a single topical pharmaceutical composition. The three ingredients are selected from the following:

In some embodiments, the composition comprises niacinamide, or a pharmaceutically acceptable salt thereof, and can range, e.g., from about 0.1% w/w to about 15% w/w of the composition, from about 2% w/w to about 6% w/w, or is about 2%, about 3%, about 4%, about 5%, or about 6% w/w of the composition. For example, in some embodiments, the niacinamide, or a pharmaceutically acceptable salt thereof, is about 4% w/w of the composition.

In some embodiments, the composition comprises tretinoin, or a pharmaceutically acceptable salt thereof, and can range, e.g., from about 0.001% to about 1% w/w of the composition, from about 0.001% to about 0.2% w/w of the composition, or is about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.12%, about 0.13%, about 0.14%, or about 0.15% w/w of the composition. In other embodiments, the tretinoin, or a pharmaceutically acceptable salt thereof, is about 0.003% w/w of the composition, about 0.005% w/w of the composition, about 0.007% w/w of the composition, about 0.009% w/w of the composition, about 0.010% w/w of the composition, about 0.012% w/w of the composition, about 0.015% w/w of the composition, about 0.02% w/w of the composition, about 0.025% w/w of the composition, about 0.035% w/w of the composition, about 0.05% w/w of the composition, about 0.07% w/w of the composition, about 0.1% w/w of the composition, or about 0.14% w/w of the composition. In some embodiments comprising tretinoin, the composition further comprises the antioxidant butylated hydroxytoluene (BHT).

In some embodiments, the composition comprises zinc pyrithione, or a pharmaceutically acceptable salt thereof, and can range, e.g., from about 0.01% to about 2% w/w of the composition, e.g., from about 0.1% to 0.5% w/w of the composition, or is about 0.1%, about 0.15%, about 0.2%, about 0.25%, about 0.3%, about 0.35%, about 0.4%, about 0.45%, or about 0.5% w/w of the composition. In some embodiments the zinc pyrithione, or a pharmaceutically acceptable salt thereof, is about 0.25% w/w of the composition.

In some embodiments, the composition comprises azelaic acid, or a pharmaceutically acceptable salt thereof, and can range, e.g., from about 0.1% w/w to about 15% w/w of the composition, from about 2% w/w to about 7% w/w of the composition, or is about 2%, about 3%, about 4%, about 5%, about 6%, about 7% w/w of the composition, or about 8% w/w of the composition. In some embodiments, the azelaic acid, or a pharmaceutically acceptable salt thereof, is about 5% w/w of the composition. In some embodiments, the azelaic acid, or a pharmaceutically acceptable salt thereof, is about 8% w/w of the composition.

In some embodiments, the composition comprises tranexamic acid, or a pharmaceutically acceptable salt thereof, and can range, e.g., from about 0.1% w/w to about 10% w/w of the composition, from about 0.5% w/w to about 4% w/w of the composition, or is about 0.5%, about 1%, about 1.5%, about 2%, about 2.5%, about 3%, about 3.5%, about 4%, or about 5% w/w of the composition. In some embodiments, the tranexamic acid, or a pharmaceutically acceptable salt thereof, is about 2% w/w of the composition. In some embodiments, the tranexamic acid, or a pharmaceutically acceptable salt thereof, is about 5% w/w of the composition.

In some embodiments, the composition comprises metronidazole, azelaic acid, and zinc pyrithione, or pharmaceutically acceptable salts thereof. In some embodiments, the composition consists of metronidazole, azelaic acid, and zinc pyrithione, or pharmaceutically acceptable salts thereof.

In some embodiments, the composition comprises in percent weight/weight (% w/w) three compounds including about 0.5% to about 2% metronidazole, or a pharmaceutically acceptable salt thereof, about 4% to about 6% azelaic acid, or a pharmaceutically acceptable salt thereof, and about 0.1% to about 0.5% zinc pyrithione, or a pharmaceutically acceptable salt thereof. In other embodiments, the composition consists of % w/w three compounds including about 0.5% to about 2% metronidazole, or a pharmaceutically acceptable salt thereof, about 4% to about 6% azelaic acid, or a pharmaceutically acceptable salt thereof, and about 0.1% to about 0.5% zinc pyrithione, or a pharmaceutically acceptable salt thereof.

In some embodiments, the composition comprises about 1% w/w metronidazole, about 5% w/w azelaic acid, and about 0.25% w/w zinc pyrithione, or pharmaceutically acceptable salts thereof. In some embodiments, the composition consists of about 1% w/w metronidazole, about 5% w/w azelaic acid, and about 0.25% w/w zinc pyrithione, or pharmaceutically acceptable salts thereof.

In some embodiments, the composition comprises azelaic acid, tranexamic acid, and niacinamide, or pharmaceutically acceptable salts thereof. In some embodiments, the composition consists of azelaic acid, tranexamic acid, and niacinamide, or pharmaceutically acceptable salts thereof.

In some embodiments, the composition comprises % w/w three compounds including about 4% to about 6% azelaic acid, or a pharmaceutically acceptable salt thereof, about 1% to about 4% tranexamic acid, or a pharmaceutically acceptable salt thereof, and about 2% to about 6% niacinamide, or a pharmaceutically acceptable salt thereof. In some embodiments, the composition consists of % w/w three compounds including about 4% to about 6% azelaic acid, or a pharmaceutically acceptable salt thereof, about 1% to about 4% tranexamic acid, or a pharmaceutically acceptable salt thereof, and about 2% to about 6% niacinamide, or a pharmaceutically acceptable salt thereof.

In other embodiments, the composition comprises about 5% w/w azelaic acid, about 2% w/w tranexamic acid, and about 4% w/w niacinamide, or pharmaceutically acceptable salts thereof. In some embodiments, the composition consists of about 5% w/w azelaic acid, about 2% w/w tranexamic acid, and about 4% w/w niacinamide, or pharmaceutically acceptable salts thereof.

In some embodiments, the composition comprises tretinoin, tranexamic acid, and niacinamide, or pharmaceutically acceptable salts thereof. In some embodiments, the composition consists of tretinoin, tranexamic acid, and niacinamide, or pharmaceutically acceptable salts thereof.

In certain embodiments, the composition comprises % w/w three compounds including about 0.001% to about 0.2% tretinoin, or a pharmaceutically acceptable salt thereof, about 1% to about 6% tranexamic acid, or a pharmaceutically acceptable salt thereof, and about 2% to about 6% niacinamide, or a pharmaceutically acceptable salt thereof. In other embodiments, the composition consists of % w/w three compounds including about 0.001% to about 0.2% tretinoin, or a pharmaceutically acceptable salt thereof, about 1% to about 6% tranexamic acid, or a pharmaceutically acceptable salt thereof, and about 2% to about 6% niacinamide, or a pharmaceutically acceptable salt thereof.

In some embodiments, the composition comprises about 0.003% w/w tretinoin, from about 2% to about 5% w/w tranexamic acid, and about 4% w/w niacinamide, or pharmaceutically acceptable salts thereof. In exemplary embodiments, the composition comprises about 0.003% w/w tretinoin, about 2% w/w tranexamic acid, and about 4% w/w niacinamide, or pharmaceutically acceptable salts thereof. In other embodiments, the composition comprises about 0.003% w/w tretinoin, about 5% w/w tranexamic acid, and about 4% w/w niacinamide, or pharmaceutically acceptable salts thereof. In some embodiments, the composition consists of about 0.003% w/w tretinoin, from about 2% to about 5% w/w tranexamic acid, and about 4% w/w niacinamide, or pharmaceutically acceptable salts thereof. In exemplary embodiments, the composition consists of about 0.003% w/w tretinoin, about 2% w/w tranexamic acid, and about 4% w/w niacinamide, or pharmaceutically acceptable salts thereof. In other embodiments, the composition consists of about 0.003% w/w tretinoin, about 5% w/w tranexamic acid, and about 4% w/w niacinamide, or pharmaceutically acceptable salts thereof.

In some embodiments, the composition comprises about 0.005% w/w tretinoin, from about 2% to about 5% w/w tranexamic acid, and about 4% w/w niacinamide, or pharmaceutically acceptable salts thereof. In exemplary embodiments, the composition comprises about 0.005% w/w tretinoin, about 2% w/w tranexamic acid, and about 4% w/w niacinamide, or pharmaceutically acceptable salts thereof. In other embodiments, the composition comprises about 0.005% w/w tretinoin, about 5% w/w tranexamic acid, and about 4% w/w niacinamide, or pharmaceutically acceptable salts thereof. In some embodiments, the composition consists of about 0.005% w/w tretinoin, from about 2% to about 5% w/w tranexamic acid, and about 4% w/w niacinamide, or pharmaceutically acceptable salts thereof. In exemplary embodiments, the composition consists of about 0.005% w/w tretinoin, about 2% w/w tranexamic acid, and about 4% w/w niacinamide, or pharmaceutically acceptable salts thereof. In other embodiments, the composition consists of about 0.005% w/w tretinoin, about 5% w/w tranexamic acid, and about 4% w/w niacinamide, or pharmaceutically acceptable salts thereof.

In some embodiments, the composition comprises about 0.007% w/w tretinoin, from about 2% to about 5% w/w tranexamic acid, and about 4% w/w niacinamide, or pharmaceutically acceptable salts thereof. In exemplary embodiments, the composition comprises about 0.007% w/w tretinoin, about 2% w/w tranexamic acid, and about 4% w/w niacinamide, or pharmaceutically acceptable salts thereof. In other embodiments, the composition comprises about 0.007% w/w tretinoin, about 5% w/w tranexamic acid, and about 4% w/w niacinamide, or pharmaceutically acceptable salts thereof. In some embodiments, the composition consists of about 0.007% w/w tretinoin, from about 2% to about 5% w/w tranexamic acid, and about 4% w/w niacinamide, or pharmaceutically acceptable salts thereof. In exemplary embodiments, the composition consists of about 0.007% w/w tretinoin, about 2% w/w tranexamic acid, and about 4% w/w niacinamide, or pharmaceutically acceptable salts thereof. In other embodiments, the composition consists of about 0.007% w/w tretinoin, about 5% w/w tranexamic acid, and about 4% w/w niacinamide, or pharmaceutically acceptable salts thereof.

In some embodiments, the composition comprises about 0.009% w/w tretinoin, from about 2% to about 5% w/w tranexamic acid, and about 4% w/w niacinamide, or pharmaceutically acceptable salts thereof. In exemplary embodiments, the composition comprises about 0.009% w/w tretinoin, about 2% w/w tranexamic acid, and about 4% w/w niacinamide, or pharmaceutically acceptable salts thereof. In other embodiments, the composition comprises about 0.009% w/w tretinoin, about 5% w/w tranexamic acid, and about 4% w/w niacinamide, or pharmaceutically acceptable salts thereof. In some embodiments, the composition consists of about 0.009% w/w tretinoin, from about 2% to about 5% w/w tranexamic acid, and about 4% w/w niacinamide, or pharmaceutically acceptable salts thereof. In exemplary embodiments, the composition consists of about 0.009% w/w tretinoin, about 2% w/w tranexamic acid, and about 4% w/w niacinamide, or pharmaceutically acceptable salts thereof. In other embodiments, the composition consists of about 0.009% w/w tretinoin, about 5% w/w tranexamic acid, and about 4% w/w niacinamide, or pharmaceutically acceptable salts thereof.

In some embodiments, the composition comprises about 0.012% w/w tretinoin, from about 2% to about 5% w/w tranexamic acid, and about 4% w/w niacinamide, or pharmaceutically acceptable salts thereof. In exemplary embodiments, the composition comprises about 0.012% w/w tretinoin, about 2% w/w tranexamic acid, and about 4% w/w niacinamide, or pharmaceutically acceptable salts thereof. In other embodiments, the composition comprises about 0.012% w/w tretinoin, about 5% w/w tranexamic acid, and about 4% w/w niacinamide, or pharmaceutically acceptable salts thereof. In some embodiments, the composition consists of about 0.012% w/w tretinoin, from about 2% to about 5% w/w tranexamic acid, and about 4% w/w niacinamide, or pharmaceutically acceptable salts thereof. In exemplary embodiments, the composition consists of about 0.012% w/w tretinoin, about 2% w/w tranexamic acid, and about 4% w/w niacinamide, or pharmaceutically acceptable salts thereof. In other embodiments, the composition consists of about 0.012% w/w tretinoin, about 5% w/w tranexamic acid, and about 4% w/w niacinamide, or pharmaceutically acceptable salts thereof.

In some embodiments, the composition comprises about 0.015% w/w tretinoin, from about 2% to about 5% w/w tranexamic acid, and about 4% w/w niacinamide, or pharmaceutically acceptable salts thereof. In exemplary embodiments, the composition comprises about 0.015% w/w tretinoin, about 2% w/w tranexamic acid, and about 4% w/w niacinamide, or pharmaceutically acceptable salts thereof. In other embodiments, the composition comprises about 0.015% w/w tretinoin, about 5% w/w tranexamic acid, and about 4% w/w niacinamide, or pharmaceutically acceptable salts thereof. In some embodiments, the composition consists of about 0.015% w/w tretinoin, from about 2% to about 5% w/w tranexamic acid, and about 4% w/w niacinamide, or pharmaceutically acceptable salts thereof. In exemplary embodiments, the composition consists of about 0.015% w/w tretinoin, about 2% w/w tranexamic acid, and about 4% w/w niacinamide, or pharmaceutically acceptable salts thereof. In other embodiments, the composition consists of about 0.015% w/w tretinoin, about 5% w/w tranexamic acid, and about 4% w/w niacinamide, or pharmaceutically acceptable salts thereof.

In other embodiments, the composition comprises about 0.02% w/w tretinoin, from about 2% to about 5% w/w tranexamic acid, and about 4% w/w niacinamide, or pharmaceutically acceptable salts thereof. In exemplary embodiments, the composition comprises about 0.02% w/w tretinoin, about 2% w/w tranexamic acid, and about 4% w/w niacinamide, or pharmaceutically acceptable salts thereof. In other embodiments, the composition comprises about 0.02% w/w tretinoin, about 5% w/w tranexamic acid, and about 4% w/w niacinamide, or pharmaceutically acceptable salts thereof. In some embodiments, the composition consists of about 0.02% w/w tretinoin, from about 2% to about 5% w/w tranexamic acid, and about 4% w/w niacinamide, or pharmaceutically acceptable salts thereof. In exemplary embodiments, the composition consists of about 0.02% w/w tretinoin, about 2% w/w tranexamic acid, and about 4% w/w niacinamide, or pharmaceutically acceptable salts thereof. In other embodiments, the composition consists of about 0.02% w/w tretinoin, about 5% w/w tranexamic acid, and about 4% w/w niacinamide, or pharmaceutically acceptable salts thereof.

In some embodiments, the composition comprises about 0.035% w/w tretinoin, from about 2% to about 5% w/w tranexamic acid, and about 4% w/w niacinamide, or pharmaceutically acceptable salts thereof. In exemplary embodiments, the composition comprises about 0.035% w/w tretinoin, about 2% w/w tranexamic acid, and about 4% w/w niacinamide, or pharmaceutically acceptable salts thereof. In other embodiments, the composition comprises about 0.035% w/w tretinoin, about 5% w/w tranexamic acid, and about 4% w/w niacinamide, or pharmaceutically acceptable salts thereof. In some embodiments, the composition consists of about 0.035% w/w tretinoin, about 2% w/w tranexamic acid, and about 4% w/w niacinamide, or pharmaceutically acceptable salts thereof. In exemplary embodiments, the composition consists of about 0.035% w/w tretinoin, about 2% w/w tranexamic acid, and about 4% w/w niacinamide, or pharmaceutically acceptable salts thereof. In other embodiments, the composition consists of about 0.035% w/w tretinoin, about 5% w/w tranexamic acid, and about 4% w/w niacinamide, or pharmaceutically acceptable salts thereof.

In certain embodiments, the composition comprises about 0.05% w/w tretinoin, from about 2% to about 5% w/w tranexamic acid, and about 4% w/w niacinamide, or pharmaceutically acceptable salts thereof. In exemplary embodiments, the composition comprises about 0.05% w/w tretinoin, about 2% w/w tranexamic acid, and about 4% w/w niacinamide, or pharmaceutically acceptable salts thereof. In other embodiments, the composition comprises about 0.05% w/w tretinoin, about 5% w/w tranexamic acid, and about 4% w/w niacinamide, or pharmaceutically acceptable salts thereof. In some embodiments, the composition consists of about 0.05% w/w tretinoin, about 2% w/w tranexamic acid, and about 4% w/w niacinamide, or pharmaceutically acceptable salts thereof. In exemplary embodiments, the composition consists of about 0.05% w/w tretinoin, about 2% w/w tranexamic acid, and about 4% w/w niacinamide, or pharmaceutically acceptable salts thereof. In other embodiments, the composition consists of about 0.05% w/w tretinoin, about 5% w/w tranexamic acid, and about 4% w/w niacinamide, or pharmaceutically acceptable salts thereof.

In yet other embodiments, the composition comprises about 0.07% w/w tretinoin, from about 2% to about 5% w/w tranexamic acid, and about 4% w/w niacinamide, or pharmaceutically acceptable salts thereof. In exemplary embodiments, the composition comprises about 0.07% w/w tretinoin, about 2% w/w tranexamic acid, and about 4% w/w niacinamide, or pharmaceutically acceptable salts thereof. In other embodiments, the composition comprises about 0.07% w/w tretinoin, about 5% w/w tranexamic acid, and about 4% w/w niacinamide, or pharmaceutically acceptable salts thereof. In some embodiments, the composition consists of about 0.07% w/w tretinoin, about 2% w/w tranexamic acid, and about 4% w/w niacinamide, or pharmaceutically acceptable salts thereof. In exemplary embodiments, the composition consists of about 0.07% w/w tretinoin, about 2% w/w tranexamic acid, and about 4% w/w niacinamide, or pharmaceutically acceptable salts thereof. In other embodiments, the composition consists of about 0.07% w/w tretinoin, about 5% w/w tranexamic acid, and about 4% w/w niacinamide, or pharmaceutically acceptable salts thereof.

In some embodiments, the composition comprises about 0.1% w/w tretinoin, from about 2% to about 5% w/w tranexamic acid, and about 4% w/w niacinamide, or pharmaceutically acceptable salts thereof. In exemplary embodiments, the composition comprises about 0.1% w/w tretinoin, about 2% w/w tranexamic acid, and about 4% w/w niacinamide, or pharmaceutically acceptable salts thereof. In other embodiments, the composition comprises about 0.1% w/w tretinoin, about 5% w/w tranexamic acid, and about 4% w/w niacinamide, or pharmaceutically acceptable salts thereof. In some embodiments, the composition consists of about 0.1% w/w tretinoin, from about 2% to about 5% w/w tranexamic acid, and about 4% w/w niacinamide, or pharmaceutically acceptable salts thereof. In exemplary embodiments, the composition consists of about 0.1% w/w tretinoin, about 2% w/w tranexamic acid, and about 4% w/w niacinamide, or pharmaceutically acceptable salts thereof. In other embodiments, the composition consists of about 0.1% w/w tretinoin, about 5% w/w tranexamic acid, and about 4% w/w niacinamide, or pharmaceutically acceptable salts thereof.

In other embodiments, the composition comprises about 0.14% w/w tretinoin, from about 2% to about 5% w/w tranexamic acid, and about 4% w/w niacinamide, or pharmaceutically acceptable salts thereof. In some embodiments, the composition consists of about 0.14% w/w tretinoin, about 2% w/w tranexamic acid, and about 4% w/w niacinamide, or pharmaceutically acceptable salts thereof. In exemplary embodiments, the composition comprises about 0.14% w/w tretinoin, about 2% w/w tranexamic acid, and about 4% w/w niacinamide, or pharmaceutically acceptable salts thereof. In other embodiments, the composition comprises about 0.14% w/w tretinoin, about 5% w/w tranexamic acid, and about 4% w/w niacinamide, or pharmaceutically acceptable salts thereof.

In some embodiments, the composition comprises tretinoin, tranexamic acid, and azelaic acid, or pharmaceutically acceptable salts thereof. In some embodiments, the composition consists of tretinoin, tranexamic acid, and azelaic acid, or pharmaceutically acceptable salts thereof.

In certain embodiments, the composition comprises % w/w three compounds including about 0.01% to about 0.05% tretinoin, or a pharmaceutically acceptable salt thereof, about 1% to about 8% tranexamic acid, or a pharmaceutically acceptable salt thereof, and about 6% to about 10% azelaic acid, or a pharmaceutically acceptable salt thereof. In other embodiments, the composition consists of % w/w three compounds including about 0.01% to about 0.05% tretinoin, or a pharmaceutically acceptable salt thereof, about 1% to about 8% tranexamic acid, or a pharmaceutically acceptable salt thereof, and about 6% to about 10% azelaic acid, or a pharmaceutically acceptable salt thereof.

In some embodiments, the composition comprises about 0.025% w/w tretinoin, about 8% w/w azelaic acid, and about 2% w/w tranexamic acid, or pharmaceutically acceptable salts thereof. In some embodiments, the composition consists of about 0.025% w/w tretinoin, about 8% w/w azelaic acid, and about 2% w/w tranexamic acid, or pharmaceutically acceptable salts thereof. In some embodiments, the composition comprises about 0.025% w/w tretinoin, about 8% w/w azelaic acid, and about 5% w/w tranexamic acid, or pharmaceutically acceptable salts thereof. In some embodiments, the composition consists of about 0.025% w/w tretinoin, about 8% w/w azelaic acid, and about 5% tranexamic acid, or pharmaceutically acceptable salts thereof.

In some embodiments, the composition comprises tretinoin, azelaic acid, and niacinamide, or pharmaceutically acceptable salts thereof. In some embodiments, the composition consists of tretinoin, azelaic acid, and niacinamide, or pharmaceutically acceptable salts thereof.

In certain embodiments, the composition comprises % w/w three compounds including about 0.001% to about 0.2% tretinoin, or a pharmaceutically acceptable salt thereof, about 1% to about 6% azelaic acid, or a pharmaceutically acceptable salt thereof, and about 2% to about 6% niacinamide, or a pharmaceutically acceptable salt thereof. In other embodiments, the composition consists of % w/w three compounds including about 0.001% to about 0.2% tretinoin, or a pharmaceutically acceptable salt thereof, about 1% to about 6% azelaic acid, or a pharmaceutically acceptable salt thereof, and about 2% to about 6% niacinamide, or a pharmaceutically acceptable salt thereof.

In some embodiments, the composition comprises about 0.003% w/w tretinoin, about 2% w/w azelaic acid, and about 4% niacinamide, or pharmaceutically acceptable salts thereof. In some embodiments, the composition consists of about 0.003% w/w tretinoin, about 2% w/w azelaic acid, and about 4% w/w niacinamide, or pharmaceutically acceptable salts thereof.

In some embodiments, the composition comprises about 0.005% w/w tretinoin, about 2% w/w azelaic acid, and about 4% w/w niacinamide, or pharmaceutically acceptable salts thereof. In some embodiments, the composition consists of about 0.005% w/w tretinoin, about 2% w/w azelaic acid, and about 4% w/w niacinamide, or pharmaceutically acceptable salts thereof.

In some embodiments, the composition comprises about 0.007% w/w tretinoin, about 2% w/w azelaic acid, and about 4% w/w niacinamide, or pharmaceutically acceptable salts thereof. In some embodiments, the composition consists of about 0.007% w/w tretinoin, about 2% w/w azelaic acid, and about 4% w/w niacinamide, or pharmaceutically acceptable salts thereof.

In some embodiments, the composition comprises about 0.01% w/w tretinoin, about 2% w/w azelaic acid, and about 4% w/w niacinamide, or pharmaceutically acceptable salts thereof. In some embodiments, the composition consists of about 0.01% w/w tretinoin, about 2% w/w azelaic acid, and about 4% w/w niacinamide, or pharmaceutically acceptable salts thereof.

In some embodiments, the composition comprises about 0.012% w/w tretinoin, about 5% w/w azelaic acid, and about 4% w/w niacinamide, or pharmaceutically acceptable salts thereof. In some embodiments, the composition consists of about 0.012% w/w tretinoin, about 5% w/w azelaic acid, and about 4% w/w niacinamide, or pharmaceutically acceptable salts thereof.

In some embodiments, the composition comprises about 0.015% w/w tretinoin, about 5% w/w azelaic acid, and about 4% w/w niacinamide, or pharmaceutically acceptable salts thereof. In some embodiments, the composition consists of about 0.015% w/w tretinoin, about 5% w/w azelaic acid, and about 4% w/w niacinamide, or pharmaceutically acceptable salts thereof.

In some embodiments, the composition comprises about 0.1% w/w tretinoin, about 5% w/w azelaic acid, and about 4% w/w niacinamide, or pharmaceutically acceptable salts thereof. In some embodiments, the composition consists of about 0.1% w/w tretinoin, about 5% w/w azelaic acid, and about 4% w/w niacinamide, or pharmaceutically acceptable salts thereof.

In certain embodiments, the composition further comprises a pharmaceutically acceptable vehicle and/or one or more inactive ingredients. At least one inactive ingredient can be a pharmaceutically acceptable vehicle. In some embodiments, at least one inactive ingredient is a preservative. In some embodiments, the composition further comprises at least one pharmaceutically acceptable vehicle, at least one emulsifier, at least one glycol, and at least one preservative. In certain embodiments, the composition further comprises one or more of the following inactive ingredients: water, vegetable glycerin, stearic acid, myristyl myristate, cetearyl alcohol, ceteareth-20, glyceryl stearate, jojoba seed oil, soybean oil, cetyl alcohol, carbomer, shea butter, calendula flower oil, passion fruit seed oil, rice bran oil, acai palm fruit oil, phenoxyethanol, ethylhexylglycerin. In one embodiment, the composition further comprises the following inactive ingredients water, aloe barbadensis leaf juice, C13-14 isoparaffin, caprylic/capric triglyceride, laureth-7, maltodextrin, phenoxyethanol, polyacrylamide, tocopheryl acetate, and triethylene glycol.

In some embodiments, the three anti-acne or anti-photoaging compounds are active at the same pH or in the same pH range. In other embodiments, the pH of the composition ranges, e.g., from about 3.0 to about 7.0. In some embodiments, the pH of the composition ranges, e.g., from about 3.5 to about 6.0. In some embodiments, the pH of the composition ranges, e.g., from about 4.0 to about 5.0.

The compositions according to the disclosure can be made as follows:

In some embodiments, the composition batch size ranges, e.g., from about 5 g to about 100 kg. In some embodiments, the composition batch size ranges, e.g., from about 100 g to about 10 kg. In some embodiments, the composition batch size ranges, e.g., from about 0.5 kg to about 3 kg. In certain embodiments, the composition batch is divided into 30 g aliquots.

In some embodiments, the composition batch size can range, e.g., from about 5 mL to about 100 L, from about 100 mL to 10 L, or from about 0.5 L to 3 L. In certain embodiments, the composition batch is divided into 30 mL aliquots.

### C. Methods of Treatment

In another aspect, the disclosure provides a method for the treatment of a skin disorder such as, but not limited to, acne, photoaging, wrinkles and/or uneven pigmentation in a subject in need thereof, comprising administering to the skin of the subject a composition according to the disclosure.

In some embodiments, the composition is administered topically. The topical compositions of the present disclosure can be provided in the form of a cream (ointment), a gel, or lotion. The pharmaceutically acceptable vehicle is selected according to the desired final form of the topical composition (cream or ointment, gel, lotion, and the like) from the types of vehicles known in the art for topical application of active ingredients.

According to some embodiments, administration of the topical pharmaceutical composition to the skin of a subject results in reduction of fine lines and wrinkles, reduction in acne, skin firming, improvement in skin texture, improvement in the skin's elasticity, improvement in skin luminosity, reduction of uneven pigmentation, skin hydration, skin moisturization, reduction in skin dehydration, and improvement of even skin tone.

In some embodiments, the frequency of application of the disclosed compositions to the skin of a subject is determined by a medical provider. In some embodiments, the frequency of application of the disclosed compositions to the skin of a subject may be one, two, or three times per day. In certain embodiments, the frequency of application is once per day. In some embodiments the application of the disclosed compositions to the skin of a subject occurs at night. In certain embodiments, the application of the disclosed compositions occurs before the subject goes to sleep.

According to some embodiments, the method includes evaluating the skin of a subject, such as, but not limited to, by using telemedicine. According to some embodiments, a first topical pharmaceutical composition is administered to the subject. The skin of the treated subject may then be reevaluated. According to some embodiments, if the skin evaluated has not improved, a second topical pharmaceutical composition is administered. The second topical pharmaceutical composition may comprise ingredients that cause enhanced skin irritation when compared to the first topical pharmaceutical composition.

In some embodiments, the treated skin of the subject is then evaluated for skin brightness, discoloration, fine lines, and/or wrinkles. In certain embodiments, evaluating the skin of the subject includes one or more of the following: creation and use of a portal (e.g., through the internet), which allows secure examination of high-resolution photographs; remote examination of high-resolution photographs; and in person examination by a qualified grader. In other embodiments, the skin of the subject is evaluated via a form of telemedicine via secure uploading of the subject's medical history and digital high-resolution photographs online or through the internet. In still other embodiments, evaluating the skin of the subject includes evaluating skin by profilometry; evaluating skin tone; evaluating skin color; evaluating skin firmness; evaluating skin elasticity; evaluating skin hydration; and evaluating skin aging by visual assessments. In some embodiments, evaluating the skin of the subject includes one or more of the following: profilometric analysis; measurements with a CUTOMETER® MPA 580; measurements with a CHROMAMETER CR300®; measurements with a CORNEOMETER® CM825; expert visual assessments; and visual assessments with Visia-CR® Capture.

In some embodiments, the expected effects of the treatment including, but non- limited to, visible reduction of fine lines and wrinkles, skin firming, improvement in skin texture, improvement in the skin's elasticity, improvement in skin luminosity, reduction of uneven pigmentation, hydrating, moisturizing, combating skin dehydration, and/or encouraging even skin tone, are evaluated after an interval of time.

For example, improvement of acne, e.g., less acne, or of photoaging, e.g., less discoloration, fine lines, and/or wrinkles, is evaluated after an interval of time. The interval of time can range, e.g., from about 1 day to about a year, or from about 1 week to about 6 months (e.g. about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks). In one embodiment, the first interval is four weeks. In other embodiments, evaluating the skin of the subject occurs at one or more intervals of time over the course of treatment. In some embodiments, the method of treatment of acne and photoaging in a subject in need thereof is applied over a period of time that can range, e.g., from about 1 day to about 50 years, or from about 1 week to about 25 years (e.g., about 3 months, about 6 months, about 1 year, about 5 years, or about 10 years).

In certain embodiments, two or more compositions are administered to the skin of the subject in a method for the treatment of acne and photoaging in a subject in need thereof. In some embodiments, the skin of the subject is evaluated after an interval of time, and a different composition is administered to the subject. In some embodiments, a second composition is administered to the subject after the first composition, wherein the second composition comprises tretinoin. In some embodiments, a third composition is administered to the subject after the second composition, wherein the second and third compositions comprises tretinoin. In some embodiments, the composition comprises a higher concentration of tretinoin than previously administered to the subject. For example, the concentration of tretinoin in the first composition can be between about 0% and about 0.1% (e.g., about 0%, about 0.003%, about 0.005%, about 0.007%, about 0.009%, about 0.010%, about 0.012%, about 0.015%, about 0.020%, about 0.035%, about 0.050%, about 0.070%, and about 0.100%) of the composition w/w. In exemplary embodiments, the concentration of tretinoin in the second and/or third composition can be between about 0.003% and about 0.14% (e.g., about 0%, about 0.003%, about 0.005%, about 0.007%, about 0.009%, about 0.010%, about 0.012%, about 0.015%, about 0.020%, about 0.035%, about 0.050%, about 0.070%, about 0.100%, and about 0.14%) of the composition w/w. In some embodiments, the increase in the concentration of tretinoin may not be strictly sequential, that is, any composition comprising a higher concentration of tretinoin than the concentration of tretinoin previously administered to the subject may be used (e.g., from about 0% to about 0.015%, from about 0.005% to about 0.100%, from about 0.005 to about 0.14% of the composition w/w).

In some embodiments, evaluating the skin of the subject is based on adverse skin reactions to the tretinoin, wherein if an adverse skin reaction is not observed, a higher percentage of tretinoin is used in the second composition then was used in the first composition. In some embodiments, a higher percentage of tretinoin is used in the third composition than was used in the second composition. In some embodiments, the adverse reactions comprise redness, dryness, skin sensitivity, and inflammation.

In some embodiments, the first, second and/or third composition administered to the subject comprises azelaic acid. In some embodiments, the second and third compositions comprise a higher concentration of azelaic acid than previously administered to the subject. For example, the concentration of azelaic acid in the first composition can be between about 1% and about 3% (e.g., about 1%, about 2%, and about 3%) of the composition w/w. In exemplary embodiments, the concentration of azelaic acid in the second and/or third composition can be between about 4% and about 6% (e.g., about 4%, about 5%, and about 6%) of the composition w/w. In an exemplary embodiment, the first composition comprises about 2% w/w azelaic acid and the second composition comprises about 5% w/w azelaic acid. In another exemplary embodiment, the first composition comprises about 0% w/w azelaic acid, the second composition comprises about 2% w/w azelaic acid, and the third composition comprises about 5% w/w azelaic acid.

In some embodiments, evaluating the skin of the subject is based on adverse skin reactions to the azelaic acid, wherein if an adverse skin reaction is not observed, a higher percentage of azelaic acid is used in the second composition then was used in the first composition. In some embodiments, a higher percentage of azelaic acid is used in the third composition than was used in the second composition. In some embodiments, the adverse reactions comprise redness, dryness, skin sensitivity, and inflammation.

In some embodiments, the first, second and/or third composition administered to the subject comprises tranexamic acid. In some embodiments, the second and third compositions comprise a higher concentration of tranexamic acid than previously administered to the subject. For example, the concentration of tranexamic acid in the first composition can be between about 1% and about 3% (e.g., about 1%, about 2%, and about 3%) of the composition w/w. In exemplary embodiments, the concentration of tranexamic acid in the second and/or third composition can be between about 4% and about 6% (e.g., about 4%, about 5%, and about 6%) of the composition w/w.

In some embodiments, evaluating the skin of the subject is based on adverse skin reactions to the tranexamic acid, wherein if an adverse skin reaction is not observed, a higher percentage of tranexamic acid is used in the second composition then was used in the first composition. In some embodiments, a higher percentage of tranexamic acid is used in the third composition than was used in the second composition. In some embodiments, the adverse reactions comprise redness, dryness, skin sensitivity, and inflammation.

In other embodiments, the acne is caused by P. acnes, and the antimicrobial may target one or more of P. acnes, Staphylococcus aureus, and Staphylococcus epidermis. In other embodiments, the acne is caused by Demodex folliculorum, and metronidazole is used to target Demodex folliculorum. In some embodiments, the acne to be treated is non-inflammatory acne, also known as comedones, (e.g., blackheads and white heads). In other embodiments, the acne is inflammatory acne (e.g., papules, pustules, nodules, and cysts). In still other embodiments, the acne is a combination of non-inflammatory acne and inflammatory acne.

In certain embodiments, the acne may be classified by its severity. When a subject has several comedones but very few papules and pustules, then the subject has mild acne. If a subject has a mix of comedones and several inflamed papules and pustules existing together, the acne is mild to moderate acne. If a subject also has some nodules along with papules and pustules, the acne is moderate acne. Deep cysts or any type of acne that leaves behind permanent pitted or saucer-shaped scars is categorized as severe acne. In certain embodiments, evaluating the skin of the subject includes evaluating the severity of the acne.

A physician, clinician, or scientist having ordinary skill in the art can readily determine and prescribe the effective amount of the pharmaceutical composition required. For example, the physician, clinician, or scientist could start doses of the pharmaceutical compounds of the disclosure at levels lower than that required in order to achieve the desired therapeutic effect and gradually increase the dosage until the desired effect is achieved.

It will be understood by those having ordinary skill in the art that the specific dose level and frequency of dosage for any particular patient or subject may be varied and will depend upon a variety of factors including the activity of the specific composition employed, the metabolic stability and length of action of that composition, the age, body weight, general health, gender, diet, and the severity of the particular dermatological condition being treated. In addition, specific dose level and frequency of dosage for any particular patient also may depend on factors including, but not limited to, skin sensitivity, other medications, acne type, allergies, and prior experiences with dermatologic treatments. For example, some patients may be treated using methods of this disclosure over a period of years if the acne and/or photoaging is a chronic condition.

### D. Kits

In additional aspects, pharmaceutical kits are provided. The kit includes a sealed container approved for the storage of pharmaceutical compositions and containing one of the above-described pharmaceutical compositions. In some embodiments, the sealed container minimizes the contact of air with the ingredients, e.g. an airless bottle. In other embodiments, the sealed container is a sealed tube. In certain embodiments, the sealed container is not a pump bottle. An instruction for the use of the composition and the information about the composition are to be included in the kit.

The following examples are provided to further elucidate the advantages and features of the present application, but are not intended to limit the scope of the application. The examples are for the illustrative purposes only. USP pharmaceutical grade products were used in preparing the formulations described below.

### EXAMPLE 1

### Preparing a Pharmaceutical Composition

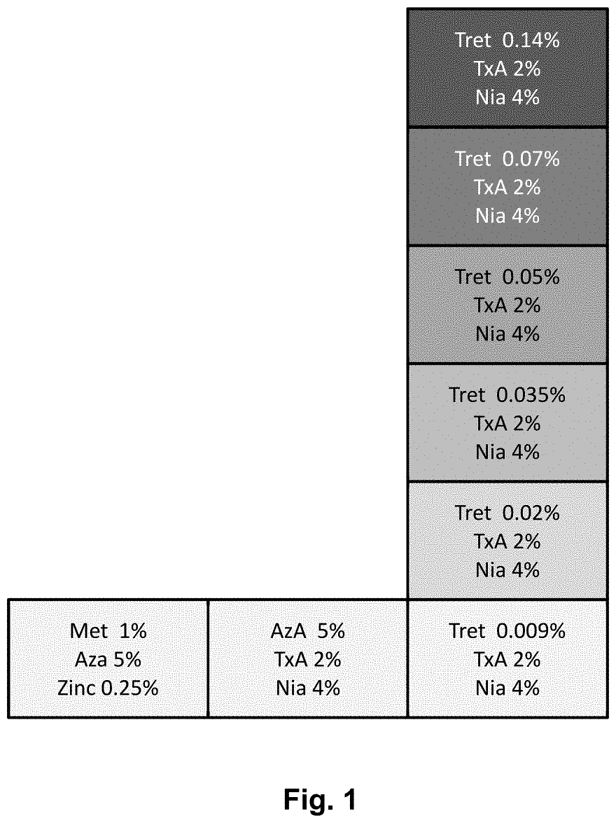

Compositions according to the disclosure comprising three distinct pharmaceutical ingredients were prepared. In FIG. 1, the percentage values of each of the three components correspond to the amount of the active ingredient in % w/w of the composition. The shading of FIG. 1 corresponds to the potential of the composition to cause irritation to the skin of the subject, with darker grays corresponding to a higher potential to cause irritation. In FIG. 1, the abbreviations used are as follows: azelaic acid (AzA); niacinamide (Nia); tretinoin (Tret); metronidazole (Met); tranexamic acid (TxA); and zinc pyrithione (Zinc).

The compositions of FIG. 1 can also include the following additional inactive ingredients: water, aloe barbadensis leaf juice, C13-14 isoparaffin, caprylic/capric triglyceride, laureth-7, maltodextrin, phenoxyethanol, polyacrylamide, tocopheryl acetate, triethylene glycol, and butylated hydroxytoluene (BHT) Antioxidant.

### EXAMPLE 2

### Evaluating the Skin by Profilometry

The skin of the subjects with acne and/or photoaging treated with pharmaceutical compositions of the disclosure are evaluated based on profilometry. Subjects are positioned on their backs with their head in line with the midline of their body. They are asked to close their eyes and maintain a neutral expression. Test sites on the skin of the subject are located using replica locating rings ensuring that each ring lay flat on the skin. The skin is not stretched or pulled during ring placement. Each ring is placed on the left and right periorbital areas of the face with the tab directed towards the back of the head. The foam and paper portions of each ring are aligned. Subjects are asked to turn their head so that the side of the head being evaluated is as horizontal as possible. The transparency film is then placed over the subject's face. Full locating rings, with centers, are then placed onto the film exactly over the site which had been selected. Landmarks are then traced onto the film using an indelible marker pen. The film is then removed from the face and labelled to identify the subject. The film is stored in a cool, dry location until next use.

### Replica Generation

The subject is placed in an identical manner to that described above and landmarks on the transparency film are lined up with the subject's facial features. A skin marker pen is then used to make dots through the film onto the face of the subject to enable exact location of the test sites. The ring is then positioned on the face, and the replicas generated by filling the well in the center of the ring with SILFLO® (JS Davis, Hert) material. Once the replica has set completely (approximately 5 minutes), it is removed from the skin, allowed to dry skin side up for a few minutes, and then placed in a storage sleeve.

### Profilometric Analysis

The following equipment and software is used: PC: IBM® compatible Pentium® III 500 MHz with 256 MB memory running under Windows® 2000 Professional. Video: Cohu® solid state B&W camera, 50mm lens/30mm extension, Coreco® TCI; Ultra frame grabber. OPTIMAS® v6.5, Microsoft® EXCEL 2000, StatSoft® STATISTICA 6. A collimated light source directed at a 25° angle from the plane of the replica is used.

The replica is placed in a holder that fixed the direction of the tab position of the replica so that the replica could be rotated to align the tab direction normal or parallel to the incident light direction. The replicas are taken from the crow's feet area adjacent to each eye with the tab direction pointing toward the ear. The NORMAL sampling orientation provides texture measurements sensitive to the MAJOR, expression-induced lines (crow's feet). The PARALLEL sampling orientation provides texture measurements sensitive to the MINOR, fine lines. The general background gradient of light intensity is adjusted by applying a 1st order correction in the direction of the light propagation.

The shadow texture produced by the oblique lighting of the negative replica is analyzed by two types of assay methods (A and B):

### Method A

The luminance is measured along a set of 10 equal length parallel lines (passes) running across the replica parallel to the lighting direction. The variations in luminance are treated as indicative of the roughness and analyzed by the following traditional surface roughness statistics:
Rz—the average maximum difference in luminance value for five equal length segments in each of the 10 lines traversing the sample;
Ra—the average deviation of the luminance curve about the mean luminance for the same 10 lines;
The “R” parameters are reported in the units of brightness (Grey Levels) ranging from 0 to 255;
FSpace—distance between markers placed on the lines at luminance changes indicative of fine lines; and
FNum—number markers per mm placed on the lines at luminance changes indicative of fine lines.

### Method B

The replica image area is then divided into 10 equal width bands or sub-areas. The shadow-like features are detected in each of these bands according to their luminance values being less than the detection threshold. The following four parameters are determined from the detected features:
Spacing—the mean distance in millimeters between adjacent detected features (i.e., spacing between the midpoints of adjacent shadowy features);
Breadth—the average breadth in millimeters of the detected features in millimeters. This parameter is proportional to the depth of the wrinkle producing the shadow;
Shadows—percent of the sampled replica area with luminance values less than the detection threshold. This is the relative area of shadows cast by the wrinkles and fine lines in the replica; and
NumWr—the total number of features detected in the 10 bands or sub-areas used to calculate spacing and breadth.

### EXAMPLE 3

### Evaluating the Skin Firmness and Elasticity

The skin of the subjects with acne and/or photoaging treated with pharmaceutical compositions of the disclosure are evaluated based on measurements to study any changes in the viscoelastic properties of the skin as a result of treatment with the compositions of the disclosure. The measurements are performed using the CUTOMETER® MPA 580 (Courage and Khazaka, Germany). The measuring principle is based on the suction method. Negative pressure is created in the device, and the skin is drawn into the aperture of the probe. Inside the probe, the penetration depth is determined by a noncontact optical measuring system. This optical measuring system consists of a light source and a light receptor, as well as two prisms facing each other, which project the light from transmitter to receptor. The light intensity varies due to the penetration depth of the skin. The resistance of the skin to be sucked up by the negative pressure (firmness) and its ability to return into its original position (elasticity) are displayed as curves at the end of each measurement using MICROSOFT WINDOWS® based software.

### EXAMPLE 4

### Evaluating the Skin Tone and Color

The skin of the subjects with acne and/or photoaging treated with pharmaceutical compositions of the disclosure are evaluated for skin tone and color changes. Evaluation is performed using a CHROMAMETER CR300® (Courage and Khazaka, Germany) in person. The measuring head of the CR-300 uses diffuse illumination/0° viewing geometry. A pulsed xenon arc (PXA) lamp inside a mixing chamber provides diffuse, uniform lighting over the 8 mm-diameter specimen area. Only the light reflected perpendicular to the specimen surface is collected by the optical-fiber cable for color analysis. This instrument measures the amount of light reflected from the skin and quantifies this into a numerical value using the L*a*b* color scale, where L*(100) equates to total white and L*(0) equates to total black. Therefore, the L* value is inversely proportional to the Fitzpatrick visual scale of skin tone. The instrument is allowed to warm up for 30 minutes prior to use.

### EXAMPLE 5

### Evaluating the Skin Hydration

The skin of the subjects with acne and/or photoaging treated with pharmaceutical compositions of the disclosure are evaluated for humectant properties, performed using the CORNEOMETER® CM825 (Courage and Khazaka, Germany). This instrument relies on the dielectric constant, a physical property of water, which is relatively high and as such will affect the capacitance of a capacitor. Any change in the dielectric constant due to skin moisture variations will alter the capacitance of the precision capacitor in the instrument. These variations are detected electronically and are converted into a value by the CORNEOMETER®. A 15 minute warm-up period is allowed before using the CORNEOMETER®.

Prior to assessment, subjects must have been in the controlled environment (at a temperature of 22° C.±2° C. and at a relative humidity of 45%±5%) for at least 30 minutes.

Three measurements are made using the probe attachment of the CORNEOMETER® at each of the test sites on the skin of the subject, between each assessment the probe attachment of the CORNEOMETER is pressed onto a dry tissue. The next assessment is not performed until a value of 5 or less is displayed by the instrument.

### EXAMPLE 6

### Evaluating the Skin by Expert Visual Assessment

The skin of the subjects with acne and/or photoaging treated with pharmaceutical compositions of the disclosure are evaluated by the same qualified grader at each time-point for the duration of the study according to the Glogau Classification of Aging scale. Table 1 shows the skin characteristics associated with each respective classification on the Glogau Classification of Aging scale.

TABLE 1
The Glogau Classification of Aging Scale

| | Classifi- | Typical | De- | |
|---|---|---|---|---|
| Group | cation | Age | scription | Skin Characteristics |
| I | Mild | 28-35 | No | Early Photoaging: mild pigment |
| | | | wrinkles | changes, no keratosis, minimal |
| | | | | wrinkles, minimal or no makeup |
| II | Moderate | 35-50 | Wrinkles | Early to Moderate Photoaging: |
| | | | in motion | Early brown spots |
| | | | | visible, keratosis palpable |
| | | | | but not visible, parallel smile |
| | | | | lines begin to appear, wears |
| | | | | some foundation |
| III | Advanced | 50-65 | Wrinkles | Advanced Photoaging: Obvious |
| | | | at rest | discolorations, visible capillaries |
| | | | | (telangiectasias), visible keratosis, |
| | | | | wears heavier foundation always |
| IV | Severe | 60-75 | Only | Severe Photoaging: Yellow- |
| | | | wrinkles | gray skin color, prior skin |
| | | | | malignancies, wrinkles |
| | | | | throughout—no normal skin, |
| | | | | cannot wear makeup |
| | | | | because it cakes and cracks |

Illumination of the test sites on the skin of the subject is by a 60 watt pearl bulb placed approximately 30 cm from the test site on the skin of the subject.

### EXAMPLE 7

### Evaluating the Skin by Visual Assessment with Visia-CR® Capture

The skin of the subjects with acne and/or photoaging treated with pharmaceutical compositions of the disclosure are photographed and evaluated by the same qualified grader at each time-point for the duration of the study according to the Glogau Classification of Aging scale (Table 1, supra).

The Visia-CR® captures multiple lighting modalities in one computer-controlled sequence. Subjects can be photographed using standard light, UV, cross-polarization and parallel polarization techniques. The Visia-CR® is used to capture one full-face, and two side-view images (one left side and one right side), high-resolution digital image of each subject with their eyes closed. Subjects are instructed to remain in a relaxed state while photos are captured using the Visia-CR® equipment.

### EXAMPLE 8

### Clinical Study

The compositions presented in FIG. 1 are evaluated for their ability to treat acne and photoaging.

A test composition comprising 0.035% tretinoin, 2% tranexamic acid, and 4% niacinamide of the composition % w/w is evaluated in a single-blind, bi-lateral facial site, study of 20-30 subjects aged at least 35 years with aged skin, compared to an untreated facial site. The aged skin criteria that qualifies a subject for the study includes moderate hyperpigmentation and/or photoaging in the face, hands, or décolletage areas (minimum of Grade II on Glogau classification scale, see Table 1). Usage of the compositions is evaluated over a time interval of 12 weeks.

Exclusion criteria includes one or more of the following: pregnancy or lactation; inadequate or non-existent contraception (women of child bearing potential only); a current skin disease of any type apart from mild facial acne (e.g. eczema, psoriasis); heavy alcohol consumption (i.e., more than 14 units per week or 4 units a day); current use or history of repeated use of street drugs; a febrile illness lasting more than 24 hours in the six days prior to study commencement; significant past medical history of hepatic, renal, cardiac, pulmonary, digestive, hematological, neurological, locomotor or psychiatric disease; history of asthma only if requiring regular medication or hay fever that required prescription treatment in two or more of the previous three years; a history of multiple drug hypersensitivity; concurrent medication likely to affect the response to the test articles or confuse the results of the study.; known sensitivity to the test articles or their constituents including packaging materials; current treatment by a physician for allergy unless physician consulted and participation approved; participation in a skin lightening or anti-aging study in the month prior to study state date.; recent immunization (less than 10 days prior to test commencement); a medical history indicating atopy (tendency to develop allergic diseases including eczema); no microdermabrasion treatment or superficial/light chemical peel on any study site within 30 days prior to the study period; and no use of prescription skin creams containing tretinoin or use of non-prescription retinol containing skin creams.

Prohibitions and restrictions for the duration of the study include: no use of sun beds or sun lamps for the duration of the study; no immunization from ten days prior to first assessment and product usage until completion of the study; no use of anti-aging products/treatments for the duration of the study in the assessed areas other than those issued/allocated; and discontinue use of any products containing hydroquinone, glycolic acid, alpha-hydroxy acids, salicylic acid, retinol, peptides and ascorbic acid (vitamin C including derivatives) for the study duration.

Profilometry assessments are performed according to the methods described in Example 2. Profilometric assessments of the visible appearance of fine lines and wrinkles in sites treated with the test pharmaceutical compositions of the disclosure are compared with the baseline readings and are measured for in untreated control site as well. The statistical difference between the treatment sites over baseline reading and the untreated sites over baseline reading are then be determined.

CUTOMETER® assessments of skin firmness and elasticity are performed according to the methods described in Example 3 and values are compared between a treated and untreated site. CUTOMETER® assessments are performed at 4, 8, and 12 weeks since the baseline reading.

CHROMAMETER CR300® assessments of skin tone and coloration are performed according to the methods described in Example 4 and values are compared between a treated and untreated site. CHROMAMETER CR300® assessments are performed at 4, 8, and 12 weeks since the baseline reading.

CORNEOMETER® CM825 assessments of skin moisturization are performed according to the methods described in Example 5 and are compared between a treated and untreated site. CORNEOMETER® CM825 assessments are performed at 4, 8, and 12 weeks since the baseline reading.

Visual assessments are performed according to the methods described in Example 6. Visual assessments according to the Glogau scale of visible aging (Table 1) are compared between a treated and untreated site. Visual assessments are performed at 4, 8, and 12 weeks since the baseline reading.

### Equivalents

Although the invention has been described with reference to the above examples, it will be understood that modifications and variations are encompassed within the spirit and scope of the invention.

### EXAMPLE 9

### Ramp Up of Tretinoin and Azelaic Acid in Compositions for Treatment of Acne and/or Photoaging

Compositions presented in Table 2 are administered to a subject to treat acne and photoaging.

TABLE 2

| | Tretinoin | Azelaic | Niacinamide |
|---|---|---|---|
| | % w/w | Acid % w/w | % w/w |
| 1 | 0.003 | 2 | 4 |
| 2 | 0.005 | 2 | 4 |
| 3 | 0.007 | 2 | 4 |
| 4 | 0.01 | 2 | 4 |
| 5 | 0.012 | 5 | 4 |
| 6 | 0.015 | 5 | 4 |
| 7 | 0.02 | 5 | 4 |
| 8 | 0.035 | 5 | 4 |
| 9 | 0.05 | 5 | 4 |
| 10 | 0.070 | 5 | 4 |
| 11 | 0.1 | 5 | 4 |
| 12 | 0.14 | 5 | 4 |

A subject is administered a first composition from Table 2 comprising a low value of azelaic acid and tretinoin as w/w % of the composition. A test composition comprising 0.03% tretinoin, 2% azelaic acid, and 4% niacinamide of the composition % w/w is evaluated.

The skin of the subjects is evaluated based on profilometry according to the methods of Example 2. CUTOMETER® assessments of skin firmness and elasticity are performed according to the methods described in Example 3. CHROMAMETER CR300® assessments of skin tone and coloration are performed according to the methods described in Example 4. CORNEOMETER®CM825 assessments of skin moisturization are performed according to the methods described in Example 5. Visual assessments are performed according to the methods described in Examples 6 and 7.

If a subject shows skin improvements as determined by the methods of Examples 3-7 (including a decrease in wrinkle depth and area, increase in skin firmness or elasticity, decreased discoloration, or increase in skin moisturization) and does not exhibit a significant loss in skin moisture as measured according to Example 5 or a significant increase in skin redness/discoloration as measured according to the methods of Example 4, then the subject is administered a second composition comprising a higher value of azelaic acid and tretinoin as w/w % of the composition then is administered in the first composition. The subject is administered a second composition from Table 2 comprising 0.012% tretinoin, 5% azelaic acid, and 4% niacinamide of the composition % w/w.

The skin of the subjects is again evaluated based on the methods of Examples 4 and 5 and if the subject does not exhibit a significant loss in skin moisture as measured according to Example 5 or a significant increase in skin redness/discoloration as measured according to the methods of Example 4, then the subject will continue to be administered to second composition.

1. A composition comprising metronidazole, azelaic acid, and zinc pyrithione, or pharmaceutically acceptable salts thereof.

2.-3. (canceled)

4. A composition comprising azelaic acid, tranexamic acid, and niacinamide, or pharmaceutically acceptable salts thereof.

5.-6. (canceled)

7. A composition comprising tretinoin, tranexamic acid, and niacinamide, or pharmaceutically acceptable salts thereof.

8.-44. (canceled)

45. A composition comprising tretinoin, tranexamic acid, and azelaic acid, or pharmaceutically acceptable salts thereof.

46.-48. (canceled)

49. A composition comprising from about 0.003% to about 0.010% w/w tretinoin, about 2% w/w azelaic acid, and about 4% w/w niacinamide, or pharmaceutically acceptable salts thereof.

50.-52. (canceled)

53. A composition comprising from about 0.012% to about 0.1% w/w tretinoin, about 5% w/w azelaic acid, and about 4% w/w niacinamide, or pharmaceutically acceptable salts thereof.

54.-56. (canceled)

57. The composition of claim 1, further comprising a pharmaceutically acceptable vehicle.

58.-60. (canceled)

61. A method for the treatment of acne, photoaging, and/or uneven pigmentation in a subject in need thereof comprising administering to the skin of the subject the composition of claim 1.

62. (canceled)

63. A kit comprising:
a) the composition of claim 1;
b) a sealed container for housing the composition; and
c) instructions for use.

64. A method for treating acne or photoaging in a subject in need thereof comprising:
a) administering a therapeutically effective amount of a first composition to the skin of the subject, wherein the first composition comprises niacinamide, tranexamic acid, and tretinoin, or pharmaceutically acceptable salts thereof; and
b) administering a therapeutically effective amount of a second composition to the skin of the subject after a first interval after administering the first composition, wherein the second composition comprises niacinamide, tranexamic acid, and tretinoin, or pharmaceutically acceptable salts thereof, and wherein the composition comprises a higher concentration of tretinoin or tranexamic acid or both than the first composition.

65.-78. (canceled)

79. A method for treating acne or photoaging in a subject in need thereof comprising:
a) administering a therapeutically effective amount of a first composition to the skin of the subject, wherein the first composition comprises niacinamide, azelaic acid, and tretinoin or pharmaceutically acceptable salts thereof; and
b) administering a therapeutically effective amount of a second composition to the skin of the subject after a first interval after administering the first composition, wherein the second composition comprises niacinamide, azelaic acid, and tretinoin, or pharmaceutically acceptable salts thereof, and wherein the composition comprises a higher concentration of tretinoin or azelaic acid or both than the first composition.

80.-93. (canceled)

94. The composition of claim 4, further comprising a pharmaceutically acceptable vehicle.

95. A method for the treatment of acne, photoaging, and/or uneven pigmentation in a subject in need thereof comprising administering to the skin of the subject the composition of claim 4.

96. A kit comprising:
a) the composition of claim 4;
b) a sealed container for housing the composition; and
c) instructions for use.

97. The composition of claim 7, further comprising a pharmaceutically acceptable vehicle.

98. A method for the treatment of acne, photoaging, and/or uneven pigmentation in a subject in need thereof comprising administering to the skin of the subject the composition of claim 7.

99. A kit comprising:
a) the composition of claim 7;
b) a sealed container for housing the composition; and
c) instructions for use.

100. The composition of claim 45, further comprising a pharmaceutically acceptable vehicle.

101. A method for the treatment of acne, photoaging, and/or uneven pigmentation in a subject in need thereof comprising administering to the skin of the subject the composition of claim 45.

102. A kit comprising:
a) the composition of claim 45;
b) a sealed container for housing the composition; and
c) instructions for use.

103. The composition of claim 49, further comprising a pharmaceutically acceptable vehicle.

104. A method for the treatment of acne, photoaging, and/or uneven pigmentation in a subject in need thereof comprising administering to the skin of the subject the composition of claim 49.

105. A kit comprising:
a) the composition of claim 49;
b) a sealed container for housing the composition; and
c) instructions for use.

106. The composition of claim 53, further comprising a pharmaceutically acceptable vehicle.

107. A method for the treatment of acne, photoaging, and/or uneven pigmentation in a subject in need thereof comprising administering to the skin of the subject the composition of claim 53.

108. A kit comprising:
a) the composition of claim 53;
b) a sealed container for housing the composition; and
c) instructions for use.
